(19) United States
(12) Patent Application Publication
(10) Pub. No.: US 2009/0036957 A1
(43) Pub. Date: Feb. 5, 2009

(54) METHOD AND APPARATUS FOR IMPROVED PHOTON IRRADIATION THERAPY AND TREATMENT OF PAIN

(76) Inventors: Maurice Bales, Lafayette, CA (US); Leonard Mario Saputo, Orinda, CA (US); Martin James Bales, San Diego, CA (US)

Correspondence Address: REED SMITH, LLP, TWO EMBARCADERO CENTER, SUITE 2000, SAN FRANCISCO, CA, 94111, US

(21) Appl. No.: 12/236,412

(22) Filed: Sep. 23, 2008

Related U.S. Application Data
(62) Division of application No. 11/201,027, filed on Aug. 10, 2005.

(51) Int. Cl. A61N 5/06

(52) U.S. Cl. 607/90

(57) ABSTRACT

A treatment device includes a plurality of photon emitter arrays directed toward treatment points on a patient's body. The treatment points are at mirror image locations and the emitters are energized in a push-pull on-off fashion. The device is automatic, such that once a patient is inserted, an operator only need to press a single button to perform treatment. The treatment device includes, for example, fixed adjustable emitter arrays and motion control emitter arrays. A controller activates the emitter arrays according to a treatment program. The treatment program may be varied depending on patient needs and/or prescription, including length of treatment, intensity, modulation, etc. The treatment device is preferably applied to foot pain resulting from diabetic neuropathy, but may be applied to other body parts and/or ailments.

### CROSS-REFERENCE TO RELATED APPLICATIONS

This is a Divisional of U.S. patent application Ser. No. 11/201,027, filed Aug. 10, 2005 the disclosure of which is herein incorporated by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of Invention

The present invention relates to photo irradiation therapies, and more particularly to photo irradiation therapies for treating pain. The invention is yet further more related to the treatment of extremity pain, and particularly diabetic neuropathy, using an advanced photon therapy treatment device and protocol.

2. Discussion of Background

Many different Photon therapies are known and currently in use in various medical practices worldwide. Various photon therapies include advanced devices for conforming to body parts being irradiated (e.g., Van Zuylen, U.S. Pat. No. 6,221,095), therapies for stimulating acupuncture points with light irradiation (e.g., Rohlicek, U.S. Pat. No. 4,535,785), and therapies that use light of selected optical properties for maximum benefit (e.g., Salansky, U.S. Pat. Nos. 6,063,108, and 6,494,900) specific of light for optimal affect. The above noted patents and other patents and publications noted in an IDS statement submitted along with the present application are incorporated herein by reference in their entirety.

### SUMMARY OF THE INVENTION

The present inventors have realized the need for advanced regime of applying photo radiation for the treatment of pain, particularly diabetic neuropathy. The present inventors have also realize the need for a standardized treatment practice that automates treatment so that treatments may be safely and effectively administered by staff without extensive training and knowledge of the principles or theory of photon irradiation therapies (e.g., practitioners, assistants, etc), instead of a physician or specialist in photon irradiation.

In one embodiment, the present invention provides a photon irradiation device, comprising, a top photon irradiator, a bottom photon irradiator, and a control device configured to energize the top photon irradiator and the bottom photon irradiator according to a treatment protocol.

In another embodiment, the present invention provides a photon treatment device, comprising, a frame configured to accept first and second mirror image portions of a patient's body, a first photon emitter array directed toward the first mirror image portion of the patient's body, a second photon emitter array directed toward the second mirror image portion of the patient's body, and a control mechanism configured to control energization of the first and second photon emitter arrays according to a treatment protocol.

In yet another embodiment, the present invention provides a treatment method, comprising, registering a patient, fitting mirror image body parts of a patient into an automated photon array device configured to automatically irradiate treatment points on the patient's mirror image body parts, treating the patient according to a treatment protocol with the automated photon array device, and removing and debriefing the patient.

In yet another embodiment, the present invention provides a treatment control device, comprising, a controller configured to activate individual sets of photon emitter array pairs in a push-pull on-off sequence such that when a first emitter in each pair is on a second emitter in the pair is off, wherein the controller is coupled to a treatment device configured to treat mirror image body parts of a patient with each emitter pair.

Portions of both the device and method may be conveniently implemented in programming on a general purpose computer, or networked computers, and certain results or treatment logs may be displayed on an output device connected to any of the general purpose, networked computers, or transmitted to a remote device for output or display. In addition, any components of the present invention represented in a computer program, data sequences, and/or control signals may be embodied as an electronic signal broadcast (or transmitted) at any frequency in any medium including, but not limited to, wireless broadcasts, and transmissions over copper wire(s), fiber optic cable(s), and co-ax cable(s), etc.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a block diagram 100 of a treatment apparatus according to an embodiment of the present invention;

FIG. 2 is an illustration of the bottom of a patient's foot to be treated by motion control according to an embodiment of the present invention;

FIG. 3 is an illustration top of the foot above and behind the toes to be treated by either a motion control or via a fixed array according to an embodiment of the present invention;

FIG. 4 is an illustration of both sides of the rear of the foot and corresponding treatment locations 6A and 6B for the placement of a fixed array according to an embodiment of the present invention;

FIG. 5 is an illustration of a patient's lower legs and treatment locations 7A and 7B behind the popliteal artery in the knee according to an embodiment of the present invention;

FIG. 6 is an illustration of a patient's foot which shows treatment points 5A and 5B above the ankle, and treatment points 4A and 4B, below the ankle according to an embodiment of the present invention;

FIG. 7 is an illustration of a prototype photon irradiation treatment device 700 “Bigfoot,” according to an embodiment of the present invention;

FIGS. 8A-8D are drawings illustrating an adjustable multi-axis manipulator 800 according to an embodiment to the present invention;

FIG. 9 illustrates an emitter array 900 with attached heat sink 910 and fan/blower assembly 920 according to an embodiment of the present invention;

FIGS. 10A and 10B are example thermal measurements of a diabetic neuropathy patient taken before (10A) and after (10B) treatments according to an embodiment of the present invention;

FIG. 11 is a block diagram of timing and analog functions according to an embodiment of the present invention;

FIG. 12 is a block diagram of modulation and sequence devices according to an embodiment of the present invention;

FIG. 13 is a block diagram illustrating motion control and basic emitter channel control according to an embodiment of the present invention;

FIG. 14A is an illustration of an exemplary timing diagram and waveforms according to an embodiment of the present invention;

FIG. 14B is an illustration of second exemplary waveforms according to an embodiment of the present invention;

FIG. 15 is a diagram of network connections between a central office and clinics according to an embodiment of the present invention; and

FIG. 16 is a screen shot of an example web interface according to an embodiment of the present invention.

### DESCRIPTION OF THE PREFERRED EMBODIMENTS

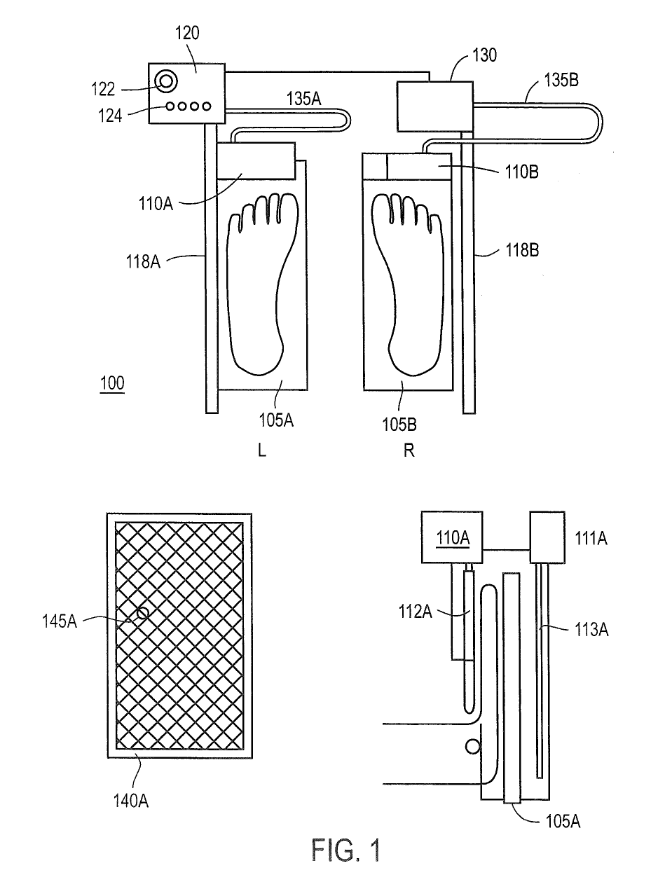

Referring now to the drawings, wherein like reference numerals designate identical or corresponding parts, and more particularly to FIG. 1 thereof, there is illustrated a block diagram 100 of a treatment apparatus according to an embodiment of the present invention. The treatment apparatus 100, and its various embodiments discussed herein is also referred to as Bigfoot. In these embodiments, the treatment apparatus 100 is specifically designed for the photon irradiation treatment of feet, and particularly treatment for diabetic neuropathy that typically manifests itself in foot pain.

The treatment apparatus 100 includes a pair of foot platforms 105 A and 105B for respectively positioning a patients Left (L) and Right (R) feet. Irradiation devices are set on a guide having a track or another guiding mechanism. For example, irradiation device 110A is set in track 112A of guide 118A.

The irradiation device 110A has a motor or other motion device that moves the irradiation device the length of the track 112A. A second irradiation device 110B is set in a second track 112B of the guide 118B. The second irradiation device 110B includes a motor or other motion device that moves the second irradiation device the length of track 112B. The irradiation devices may be coupled such that they move in tandem and powered by a single motion device. Alternatively, each of the upper (110A) and lower (111A) irradiation devices may be driven independently according to a treatment protocol that utilizes different velocities or movement patterns of the arrays. In another alternative one of the upper and lower irradiation devices are fixed arrays where each emitter is individually controlled to be on/off according to a treatment protocol. In yet another alternative, both the upper and lower arrays are fixed arrays. Second and third irradiation devices 110B and 111B (not shown) have equal, but mirror image parts that operate to irradiate a patients Right (R) foot at mirror image locations and treatment parameters consistent with the irradiation performed on the patient's Left (L) foot.

The platforms are constructed so that both top and bottom irradiation devices can simultaneously irradiate a top surface and a bottom surface of the patient's foot (e.g., irradiation device 110A (top array) irradiating the top surface of the patient's foot, and irradiation device 111A (bottom array) irradiating the bottom surface of the patient's foot). However, according to a preferred treatment program, top and bottom foot treatment occurs during different treatment time periods.

Platforms 105A and 105B provide support for the feet being irradiated. For example, platform 105A includes a frame 140A and a translucent, tennis racquet like mesh of translucent (e.g., nylon) line 145A. In another embodiment, the platform is an acrylic, glass, or other substrate transparent to the treatment quality emission from the emitter. As shown in FIG. 1, both top and bottom surfaces of the patient's feet are irradiated consistently with a treatment protocol (or program) such as, for example, one of the programs discussed further below herein.

In one embodiment, the present invention's irradiation devices are semiconductor diode arrays. The BigFoot system is comprised of thirty two (32) each of these infrared emitter arrays. The semiconductor diode arrays are, for example, configured to specifically treat diabetic neuropathy patients, and particularly foot pain associated therewith. Other forms of irradiation may also be utilized (e.g., arrays with more or less sensors, arrays of specific patterns, arrays using different photon elements (e.g., diodes, lasers, various and/or variable wavelength emitters, etc.), and others). The sensor arrays are controlled by a control module 120 that may take the form of electronics, programming, or a connection to a central database of instructions (e.g., the latest or patient customized instructions and/or treatment protocols transmitted via the Internet to a Bigfoot type device at a treatment facility).

The semiconductor diode arrays are, for example controlled as sixteen channels, which are preferably controlled as opposites or complementary. That is, when one array is on the other is off. The positions of the channel pairs (e.g., irradiation devices 110A and 110B) are opposite mirror-image body positions. That is, for each position that one emitter is placed on the body, the complementary emitter will be located on the opposite, mirror-image side. This means from the body's midline the emitters are spaced the same distance. The purpose for this arrangement is control of the sympathetic nervous system bilaterally. When an emitter is turned on it stimulates the sympathetic nerves located in the tissue below the emitter. The nerve then transfers the signal to its ganglia, adjacent to the spine, the spine then transfers the signal to the mirror-image ganglia, which in turn activates the nerves in the mirror image area of the array placement. By locating complementary arrays on the same mirror-image locations on the body and activating one array, then the complementary array the nerve signals are push-pulled from one side of the body to the opposite mirror image side having a synergistic effect with the natural response to activation of the nerves. This can help restore nerve/soft tissue blood profusion. Preferably, each channel-pair (complimentary channels) is located near an artery stimulation point because the sympathetic nerves follow and control the blood flow from the arteries to the soft tissue.

In one embodiment, the present invention utilizes a motion-controlled stage to move nine (9) emitter channels (e.g., irradiation devices 111A and 111B (not shown)) to illuminate the entire bottom or plantar surface of the feet. This was an economic choice; other array configurations could also provide appropriate stimulation and function just as well (e.g., larger or smaller arrays, or multiple individual fixed emitter arrays energized by an equivalent program). The arrays located on the motion-control stage also work as channel pairs.

For diabetic neuropathy treatment, the arrays preferably comprise 4 channels in conjunction with 4 complimentary channels. The channels are, for example, a channel placed adjacent to the entire bottom of the foot (e.g., via motion control), a channel on the top of the foot above and behind the toes (e.g., via a first fixed array), on both sides of the rear of the foot (e.g., a first side fixed array and a second side fixed array), and behind the popliteal artery in the knee (e.g., a fixed array). In one alternative, a second side of foot array placement is above the ankle. Each of the arrays may be embodied as a set of fixed arrays or a combination of fixed arrays and motion controlled arrays.

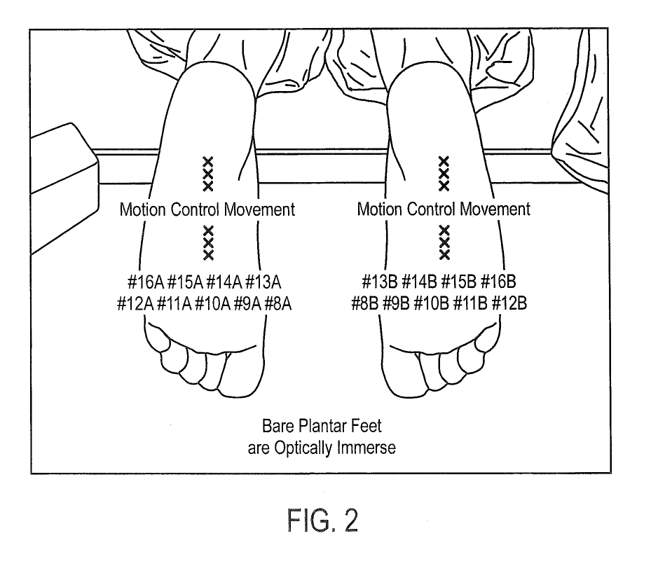

FIG. 2 is an illustration of the bottom of a patient's foot to be treated by motion control. In the directly above described embodiment, the bottom of the feet are irradiated using an array of emitters (e.g., array 111A) that is moved along the bottom of the feet so that the treatment points (e.g., illustrated treatment points, or the entire bottoms of the feet (see Table 1, for example), are irradiated in sequence according to a treatment protocol. Each foot bottom represents one channel in a pair of complimentary channels. The entire plantar feet are optically immersed.

Table 1 is a listing of treatment points according to an embodiment of the present invention. The treatment points are exemplary. Additional, less, or different treatment points may be utilized in other treatment programs.

TABLE 1

| | |
|---|---|
| #1 | On the dorsum of the foot, between the 1st and 2nd toes, |
| | proximal to the margin of the web. |
| #2 | On the dorsum of the foot, between the 2^{nd} and third toes, |
| | proximal to the margin of the web. |
| #3 | on the dorsum of the foot between 4th and 5th toe, proximal |
| | to the margin of web. |
| #4 | in the depression anterior and superior to the medial side |
| | of the tuberosity of the calcaneum. |
| #5 | directly above the tip of the medial malleolus, posterior to |
| | the border of the tibia. |
| #6 | Directly below the depression between the tip of the lateral |
| | malleolus and the Achilles tendon. |
| #7 | Mid-point of transverse popliteal crease, between tendons of |
| | bicepts femoris and semitendinosus. |
| #8-16 | Plantar feet. |

The treatment numbers are in reference to the numbers on the drawings/photos discussed further above and elsewhere herein. The treatment points are in pairs, 1A & 1B for example. These points are mirror-image points on the body. In this example embodiment, the treatment sequence is the number sequence (but again, other sequences may be utilized in differing treatment programs and not depart from the spirit and scope of the present invention).

The definitions of the treatment points listed above are the medical terms for those locations. Some of the locations are the same as acupuncture points and some are not. The total bottoms of the feet are optically immersed by use of the motion-control stage. The nine IR emitters are mounted on the motion-control stage (each side), which is moved under the feet (Plantar Feet Illumination). Other wavelength or variable wavelength emitters are an alternative.

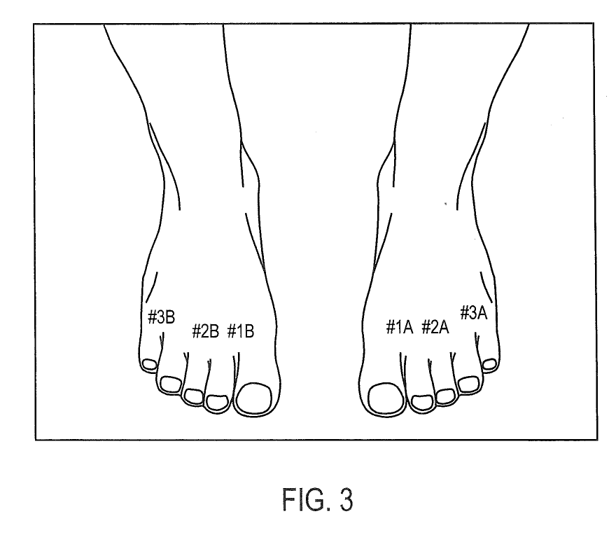

FIG. 3 is an illustration top of the foot above and behind the toes to be treated by either a motion control or via a fixed array. Each foot top represents one channel in a pair of complimentary channels. For example a fixed array may be placed over the entire top of the feet which are then irradiated by sequencing emitters above treatment points according to a treatment program. In another example, motion control is used to position an emitter array and the individual emitters of the emitter array are moved and energized (or sequenced) according to the treatment program.

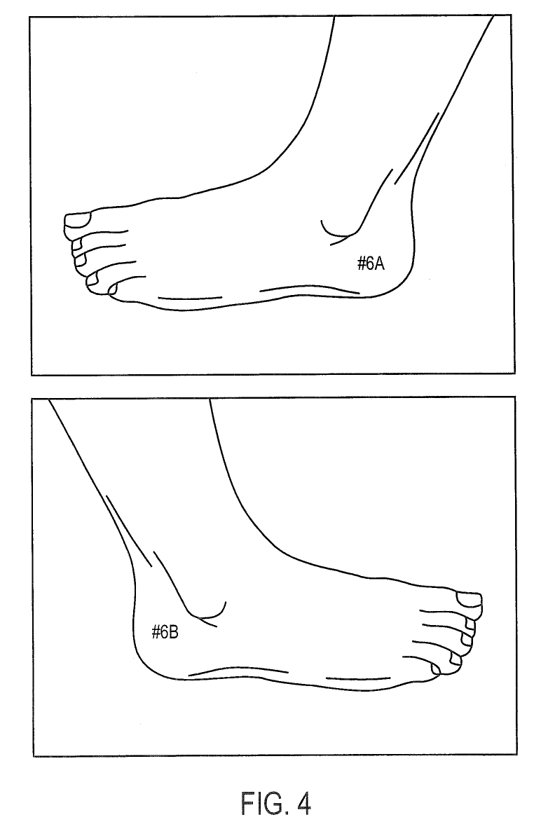

FIG. 4 is an illustration of both sides of the rear of the foot and corresponding treatment locations 6A and 6B for the placement of a fixed array. Each foot side and treatment point represents one channel in a pair of complimentary channels. The fixed array is an array of emitters that are energized (or sequenced) according to the treatment program.

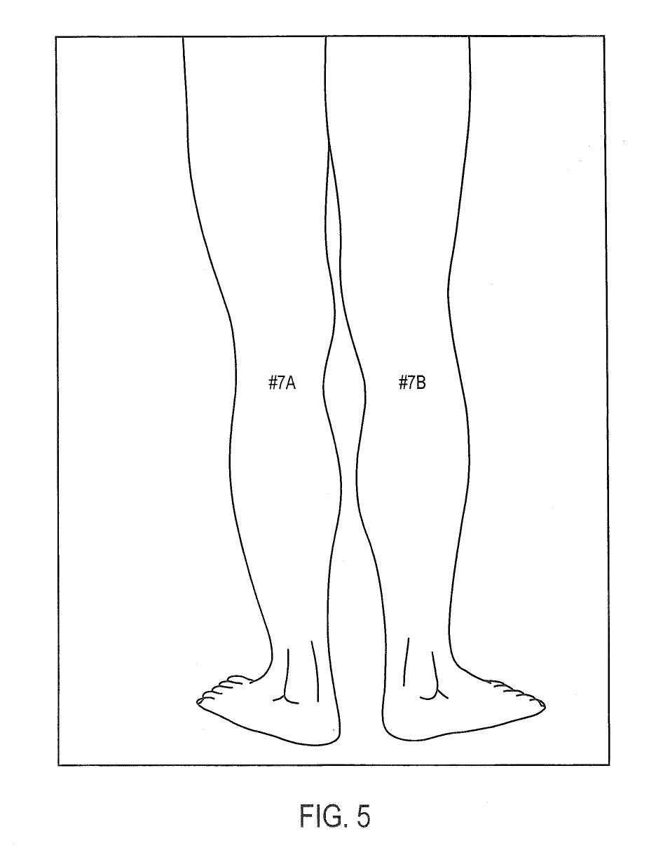

FIG. 5 is an illustration of a patient's lower legs and treatment locations 7A and 7B behind the popliteal artery in the knee. Each leg and treatment point represents one channel in a pair of complimentary channels. The treatment locations 7A and 7B are preferably treated using a fixed array positioned above the indicated location and energized according to the treatment program.

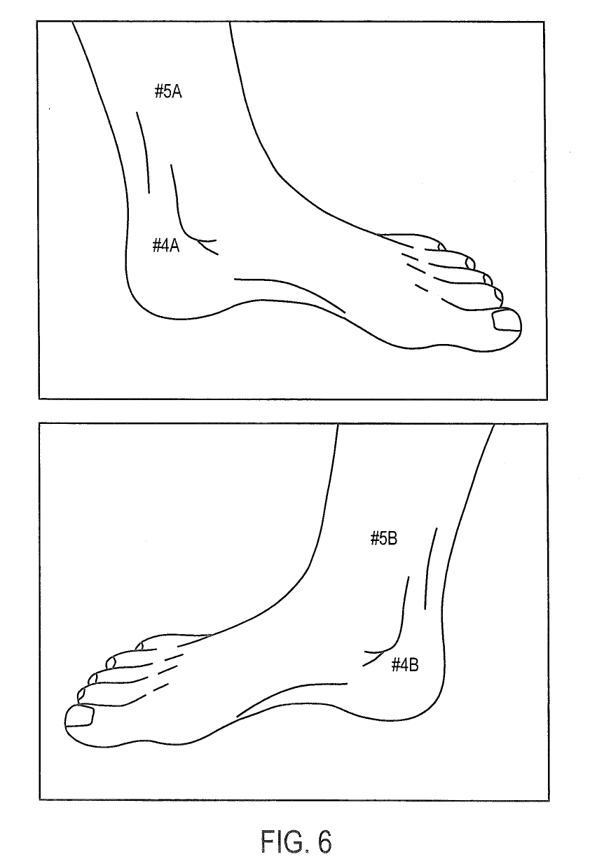

FIG. 6 is an illustration of a patient's foot and alternative treatment points 5A and 5B above the ankle, and treatment points 4A and 4B below the ankle. Each foot and treatment point represents one channel in a pair of complimentary channels. The treatment locations 5A and 5B are preferably treated using a fixed array positioned above the indicated location and energized according to the treatment program.

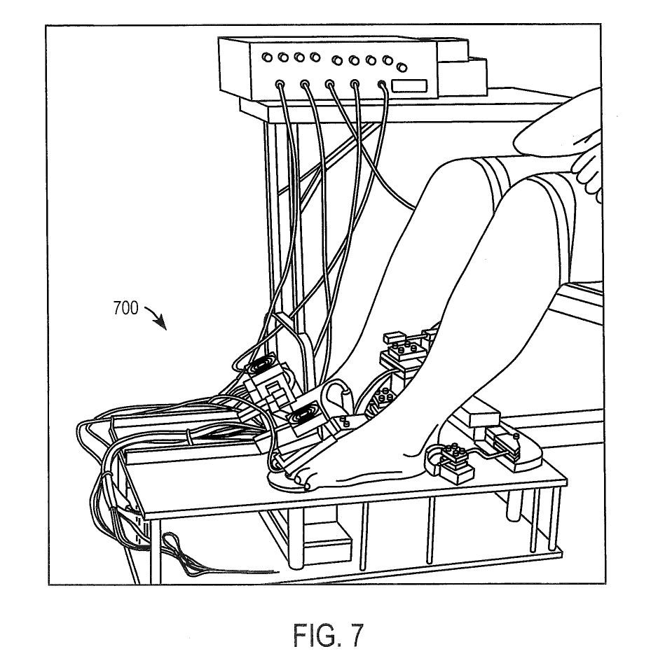

FIG. 7 is an illustration of a prototype photon irradiation treatment device 700 “Bigfoot,” according to an embodiment of the present invention. Bigfoot includes a foot platform, motion controlled emitter arrays for feet bottoms, motion controlled emitter arrays for feet tops, and fixed arrays for each of feet sides below ankle, above ankle and popliteal arteries (behind knee), and control electronics for motion control and energizing/sequencing the emitter arrays according to a treatment program for the patient.

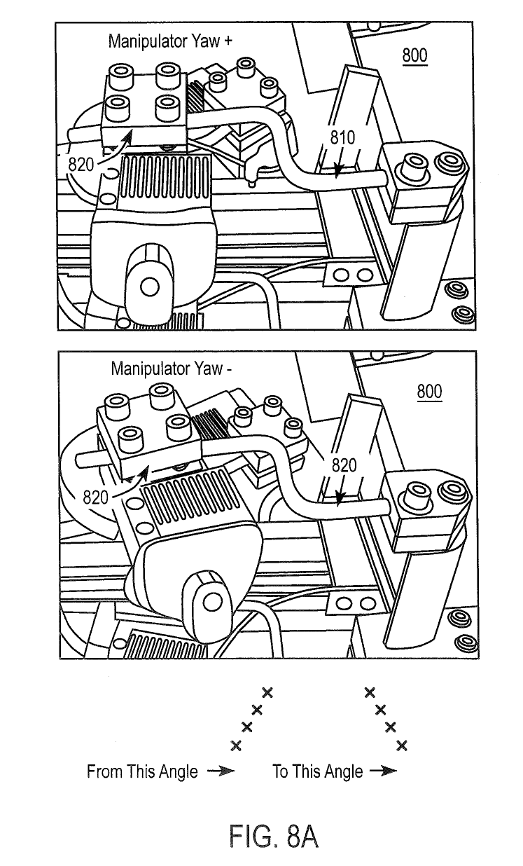

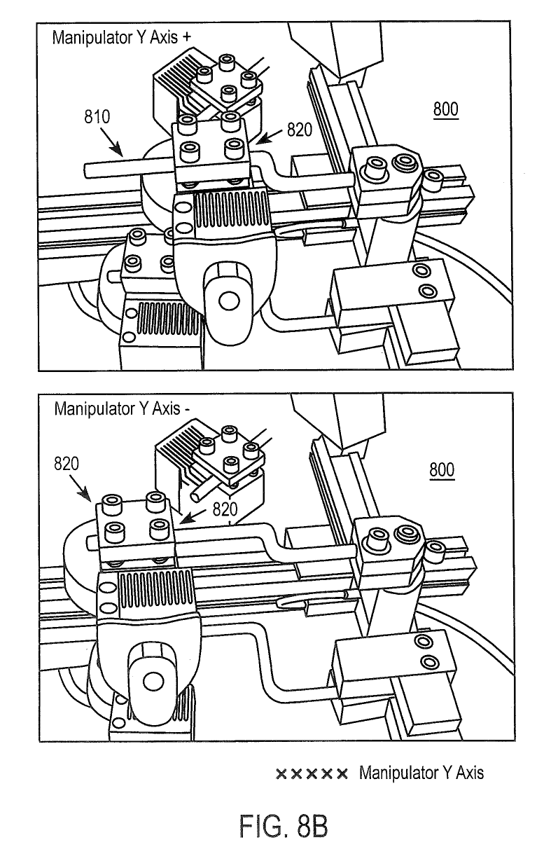

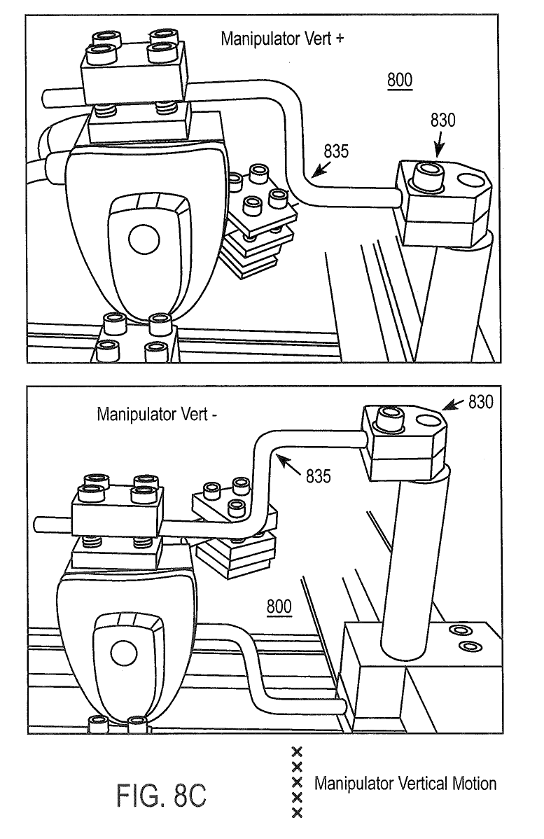

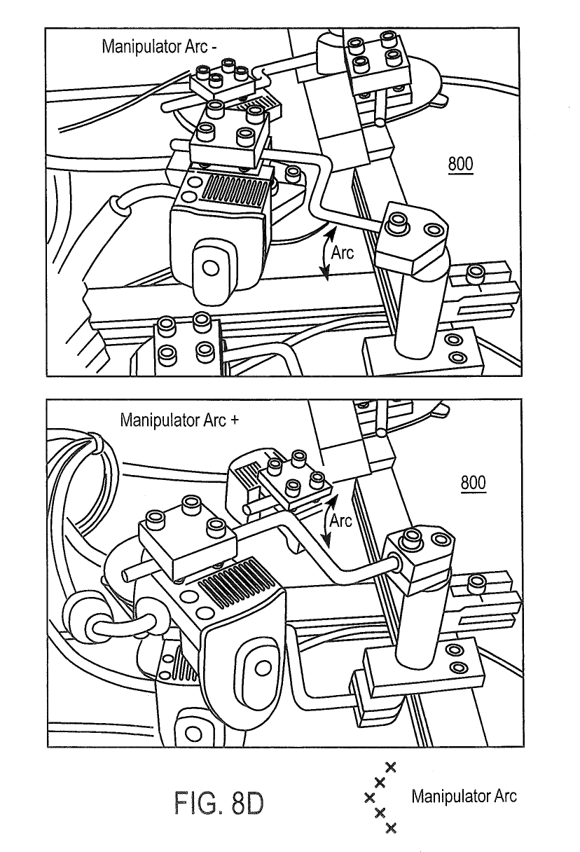

The present invention includes an adjustment mechanism, referred to as a manipulator, developed to position one or more arrays. The manipulator is used to compensate for variations in the treatment locations between varying patients. The manipulator is, for example, an adjustable positioner that is adjustable about one or more axis that enable the manipulator to easily place an emitter array at a designated treatment point without the use of tools. FIGS. 8A-8D are drawings illustrating an adjustable multi-axis manipulator 800 according to an embodiment to the present invention. The manipulator is mounted on an arm 810. The manipulator includes an arm position plate 820 that attaches an emitter array to the arm at one of a variety of angular positions. As shown in FIG. 8A, the arm position plate 820 secures the manipulator at least Yaw + and Yaw − positions. Preferably, the manipulator arm position plate has a variety of angles that the array may be positioned. As shown in FIG. 8B, the arm positioner plate 820 is slidable along the arm 810, effecting both Y axis + and Y axis − positions, and any number of positions in between along the arm 810.

As shown in FIG. 8c, the arms tension 810 is set by set screw 830, and arm 810 includes a curve 835. By rotating the arm 810, the curve adjusts a vertical position (e.g., Vert + and Vert −) of the emitter array. Once in a desired vertical position, the arm is held in place by the bushing friction. As shown in FIG. 8D, the arm is also held in an arc position by its bushing friction. The arc bushing friction holds the arm in a position along an arc (e.g., Arc −, Arc +).

Preferably, all manipulators utilize non-metallic bushings that provide adjustable friction, or otherwise constructed so each manipulator can be positioned by an operator without the need for tools.

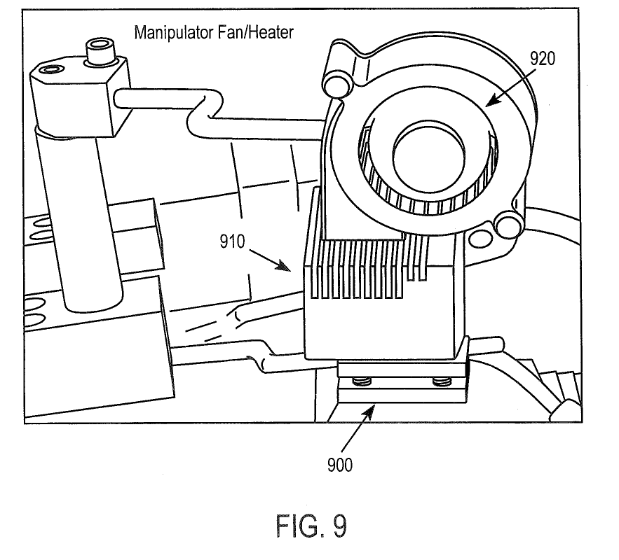

Heat dissipation of the emitter arrays is performed via one or more of a heat sink and fans attached to or in close proximity to the emitter arrays. For example, FIG. 9 illustrates an emitter array 900 with attached heat sink 910 and fan/blower assembly 920. Other devices to control heat build up in the emitters, electronics, motion control mechanisms, and in the vicinity of the patient may also be utilized, these may include the use of Thermo-electric coolers (te coolers), a solid-state heat pump device.

Returning now to FIG. 7, the photon irradiation treatment device 700, which is an example treatment device according to the present invention, is specifically designed for treatment of diabetic neuropathy as manifested in foot pain. Treatment devices according to the present invention may also be constructed for treating hands, arms, back, head, or other body parts. The device 700 includes top and bottom (motion control), side, above and below ankle (fixed), and behind knee (fixed) emitter arrays. In other devices arrays would be positioned for other or corresponding treatment points of other body parts (e.g., treatment points on the palm, back of hands, wrist, and arm for a device for treating the hands). As shown in FIG. 7, a patient's feet are positioned on a transparent platform and the fixed arrays are adjusted according to their attached manipulators to position them at their corresponding treatment points.

Once the patient's feet are positioned for irradiation (set on the platform ready for motion controlled emitter irradiation) and the fixed arrays are positioned relative to the patients treatment points, the irradiation begins according to the treatment program selected for the patient. A treatment program, for example, controls the channels in sequence. That is, one channel is on and the other channels are off (e.g., all other channels are off), then the next channel on and the other channels off and so on until each channel pair has been operational for it's time according to the treatment program. Another treatment program choice is all channels are operational simultaneously (channel pairs (e.g., complementary channel pairs), one on, the other off, then the other complementary channel on while its companion is off). In yet another embodiment, all channels corresponding to a first side of a patient are on while all channels corresponding to a second, mirror image, side of the patient are off, and vice-versa.

The emitters on the motion-controlled stage are, for example, operated in the non-sequenced mode while the stage is in motion. This is one reason for multiple emitter sequence control systems (e.g., a control system for emitters under motion control, a control system for emitters in fixed arrays, and a control system for motion of emitter arrays), channels 0-8 and 9-15.

The channels are, for example, controlled in one or more of on/off, modulation, frequency, intensity, and duration of irradiation. Combined or additional control systems may be implemented for each of these controls. The control includes, for example, a separate optical output power adjustment for each control system. The optical power adjustment choices are, for example, full, half, quarter and off e.g., power level of 5 watts max. A treatment protocol (or program) includes, for example, multi-session treatments. The protocol specifies, for example, that the power be set to half for the first treatment session. If the patient has no side effects from the first treatment then the power is set at full for the remaining treatment sessions (assuming side effects continue to be minimal or non-existant).

The Modulation, for example, can be set for External, Off, Continuous, or 72 HZ. Any frequency modulation may be applied, and, as with all other control items discussed herein, may vary between treatment protocols and patients. Preferably, for diabetic neuropathy treatment the motion-control emitter modulation is set for continuous (CH 0-8), and the fixed emitters are set for continuous.

Continuing with the example treatment protocol, the Sequence is set, for example, to off for channels 0-8 and on for channels 9-15. The sequence clock is set for 15 seconds for the sequence on channels 9-15. The channel times are set for 5 minutes for channels 0-8 and 7 minutes for channels 9-15, this provides 15 seconds on for each fixed-position emitter array.

(see FIG. 1) The Start, front panel pushbutton switch Start button 122, initiates Bigfoot's operation. When the programmed sequence is complete, for both channel groups (0-8, 9-15) then the system returns to standby. When the system is in standby depressing the Start button 122 initiates a whole new sequence. For each patient only one button needs to be pushed to initiate the treatment protocol. In one embodiment, status lights indicate progress of the treatment protocol or identify channels in operation.

The present invention may include the use of thermal imaging. Thermal imaging is utilized, for example, to provide an objective feedback on physiological changes during and after treatments. A thermal image of the feet made prior to treatment is used as a reference to compare with thermal images taken between treatments or after completion of a treatment protocol.

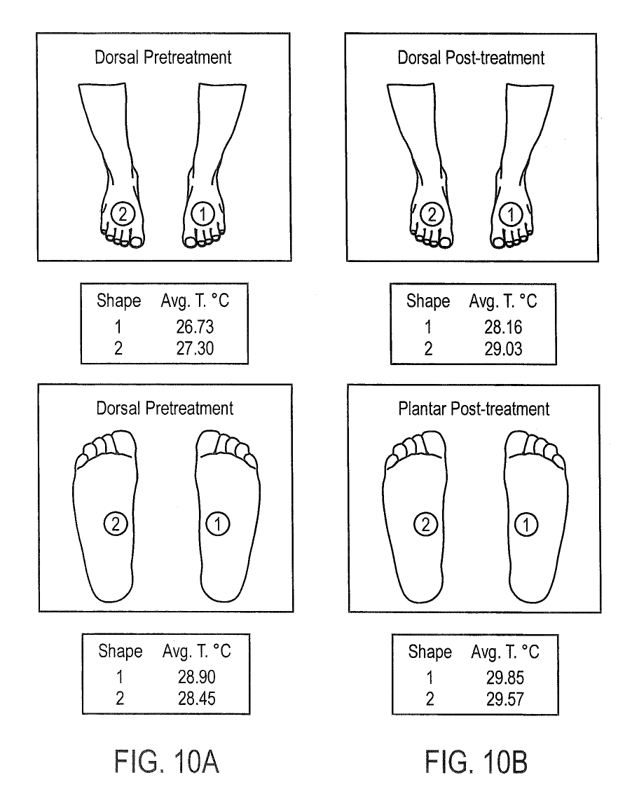

FIGS. 10A and 10B are example thermal images of a diabetic neuropathy patient taken before (10A) and after (10B) treatments according to the present invention. Thermal measurements for a patients left foot and right foot are shown for both dorsal and plantar pre-treatment (FIG. 10A), and dorsal and plantar Post-Treatment (FIG. 10B). The thermal measurements are made on the same spot of the foot pre and post treatment. The measurement is made, for example, as can be seen in the figures, at portions of the patients foot (in this example, an approximately quarter sized temperature measurement. The left and right foot temperatures are shown in degrees Celsius, and, in each case (dorsal and plantar), are elevated post treatment. The thermal measurements are indicative of blood flow/circulatory functions that have been restored/improved in the patient's feet.

The present invention includes a patient protocol. The patient protocol includes discrete individual steps that an assistant helps guide a patient through. The patient protocol is one example of a procedure that would be performed at a clinic operating according to an embodiment of the present invention.

### Possible Patient Protocol:

1. The patient arrives and fills out paperwork (e.g., basic medical, referral information, insurance information, etc.).
2. The patient arrives and disrobes from several inches above the knees to and including the feet. A patient that arrives in shorts may only need to remove shoes/socks.
3. The patient is thermal imaged.
4. The patient is positioned for Bigfoot treatment and the fixed array emitters are placed in position over their corresponding treatment points for the patient.
5. Plastic wrap is placed around treatment areas (optional).
6. The Start button on Bigfoot is depressed, treatment begins.
7. After the treatment session ends, the motion-control stage returns to home position and the emitters are deactivated.
8. The fixed array emitters are moved away from treatment areas allowing patient to be removed from Bigfoot.
9. Plastic wrap is removed and discarded.
10. Patient walks about.
11. Patient fills out paperwork and leaves.

If thermal imaging is utilized, it is normally added as pre-treatment and/or post treatment step.

In one embodiment, the fixed array emitters include a “roll out” swivel that allows the positioned fixed array emitters to be moved away from the patient to allow fast extraction of the patient without altering the relative positions of the fixed arrays. Since variations between patients is relatively minor, this allows the fixed arrays to be repositioned between patients by only making the minor variations between patients.

In one embodiment, Bigfoot control circuits are, for example, accomplished with State machines, or fixed logic. The state machine and/or fixed logic implementing control (motion control and emitter control) according to the processes and methods discussed herein. Preferably, the control circuits are implemented in programming on a general purpose computer or microprocessor (this saves patient set-up time in step 4 above).

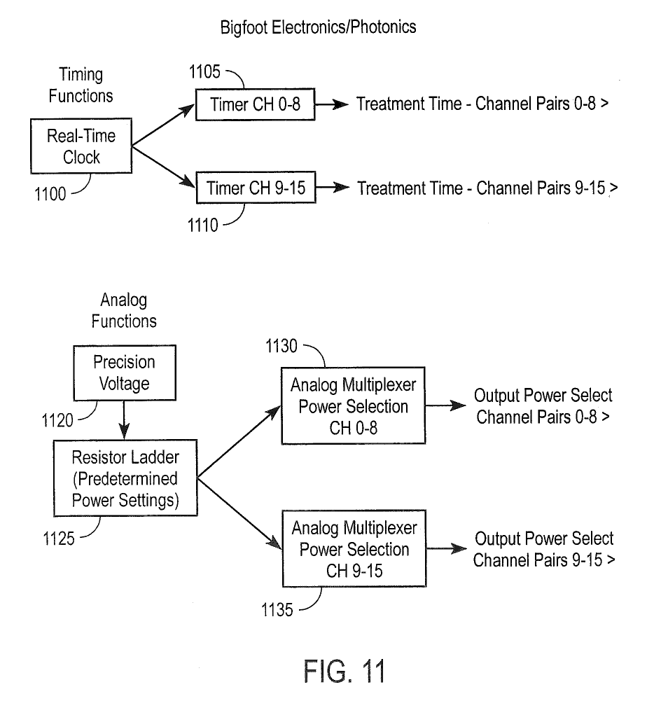

FIG. 11 is a block diagram of timing and analog functions according to an embodiment of the present invention. A real time clock (RTC) 1100 sends digital signals and is coupled to a timer 1105 that controls time of treatment on channels 0-8. A second timer 1110 also coupled to the RTC 1100 controls time of treatment on channels 9-15.

A precision voltage source 1120 provides a calibrated reference voltage for regulating channel output power. In this example embodiment, a resistance ladder 1125 predetermines the amount of power distributed to the channels. A pair of analog multiplexors 1130 and 1135 distribute the regulated power to channels 0-8 and 9-15 respectively.

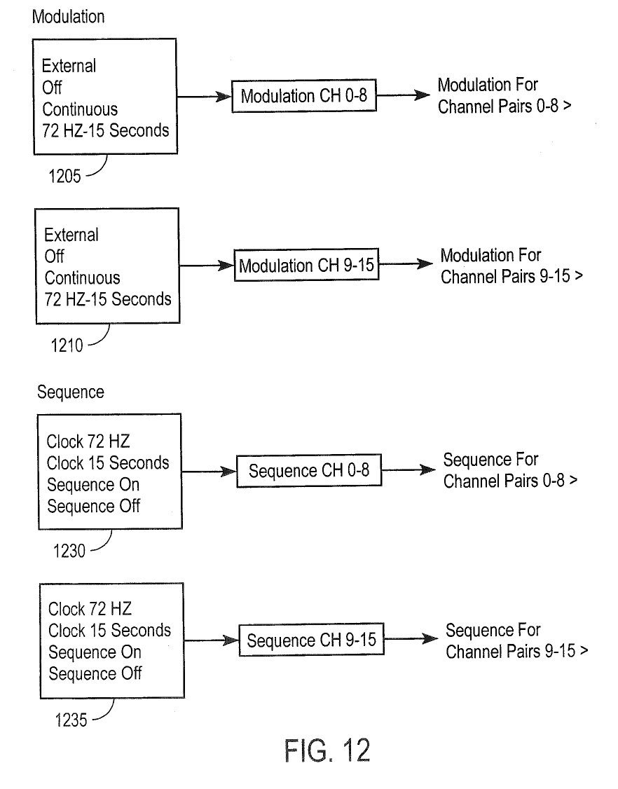

FIG. 12 is a block diagram of modulation and sequence devices according to an embodiment of the present invention. Variable (e.g., 72 Hz-15 seconds) external off continuous modulators 1205 and 1210 provide modulation for channels 0-8 and 9-15 respectively.

Sequence clocks 1230 and 1235, for example, perform modulation that is performed on channels 0-8 and 9-15 respectively.

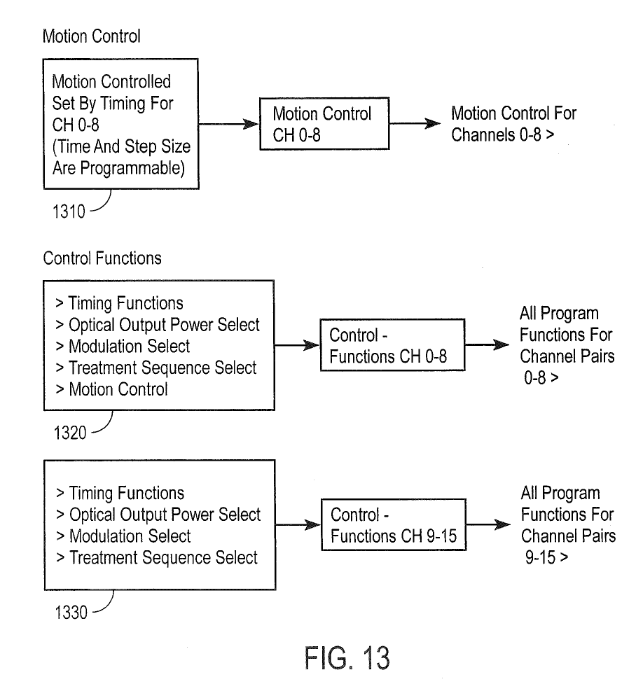

FIG. 13 is a block diagram illustrating motion control and basic emitter channel control according to an embodiment of the present invention. Motion control module 1310 prepares motion control signals used to control the motion controlled array(s) associated with, for example, channels 0-8. The motion control signals include, for example timing of motion of emitters of channels 0-8. For example, the motion of the emitter arrays is determined based on programmable features, such as duration of irradiation for each treatment point (or portions of treatment points) as specified in a treatment protocol being used for a patient.

Control functions 1320 is an example comprehensive control unit configured to implement each of timing functions, optical power output, modulation, treatment sequence (or treatment program/protocol), and motion control, all for channels 0-8. Control functions 1330 is an example comprehensive control unit configured to implement fixed array control, including timing functions, optical power, modulation, and treatment sequence. Each of the comprehensive control units may be combined or include one or more of the previously described controls, functions, modulators, etc.

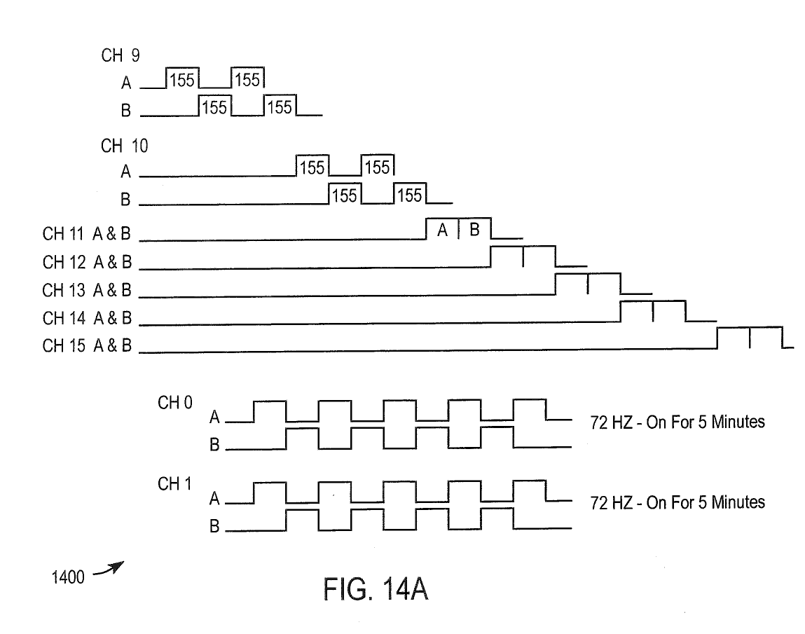

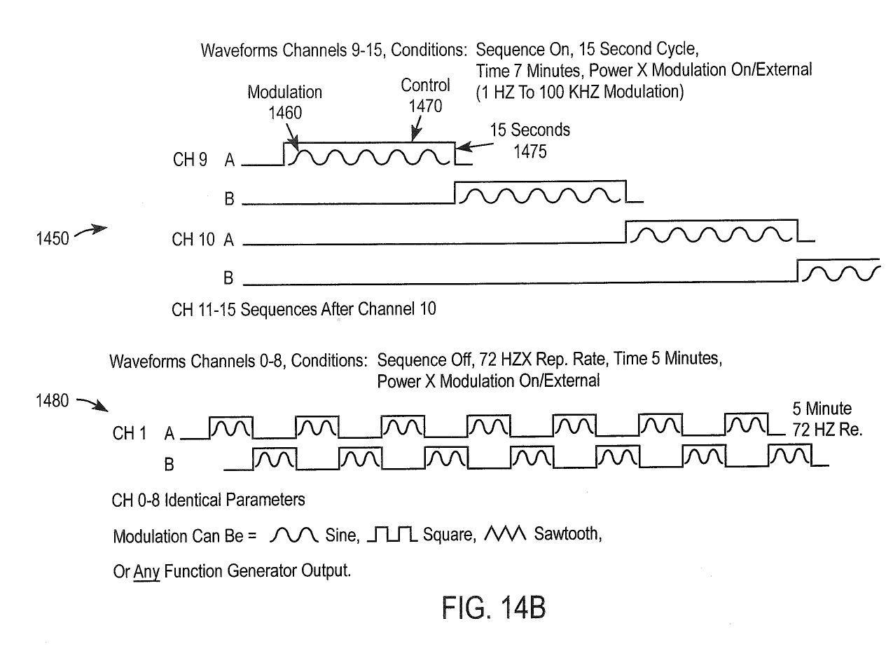

FIG. 14A is an illustration of an exemplary timing diagram and waveforms 1400 according to an embodiment of the present invention. The timing diagram includes an on-off sequence for exemplary channels 9-15. Channel 9 represents irradiation for each of complimentary channels A and B for the tops of a patient's foot. For example, the top of the right foot (channel 9, channel A) is irradiated for 15 seconds, then the top of the left foot (channel 9, complimentary channel B) is irradiated for 15 seconds. As shown in FIG. 14, the regime is then repeated. Channel 10 is then active, followed by channels 11-15 in order. All variables of the treatment, including modulation, intensity, order of irradiation, duration of each set of channel activations, pattern, and the number of repetitions may be varied and are set, for example, by a treatment program/protocol.

Channels 0-8 are intended to illustrate a waveform programmed for the patient's plantar feet illumination. Channels 0-8 are, for example, a waveform of Sequence off, 72 Hz repetition rate time 5 minutes, Power X (e.g., 1-10 watts), modulation off.

As noted above, the power output is, for example, one of 0, ¼, ½, ¾, and full. More finely set power spaces (or continuous) may also be utilized. As illustrated in FIG. 14, the settings may, for example be set such that there is one setting for channels 0-8 (channels 0 and 1 specifically illustrated, and Channels 2-8 may, for example, have identical timing of all A's and B's), and a second setting for channels 9-15. Alternatively, each channel may be independently programmable. The programmability of each channel may, for example, be set using a number of predefined programs or saved waveform regimes.

FIG. 14B is an illustration of second exemplary waveforms 1450 according to an embodiment of the present invention. The illustrated waveform 1450 comprises Sequence on, 15 second cycle, time 7 minutes, Power X, Modulation on/external. The modulation is, for example, is a set frequency between 1 Hz and 100 KHz. In one alternative, the modulation varies throughout, or parts of, the entire sequence. The illustration highlights the modulation 1460, control 1470, and duration 1475 of the waveform. A second alternative 1480, provides a Sequence off, 72 Hz repetition rate, time of 5 minutes, power X, modulation on/external for channels 0-8. In any of the example waveforms, the modulation itself may take the form of a sine wave, square wave, sawtooth, or any waveform from a function generator (e.g., a function generator output).

As noted above, the treatment protocol is, for example, a series of data that identifies the frequency, modulation, duration, intensity and other parameters applied to the photon emitters and parameters of the motion control (if any) of the treatment device. The treatment protocol may be embedded in the control electronics or programming of a treatment device according to the present invention.

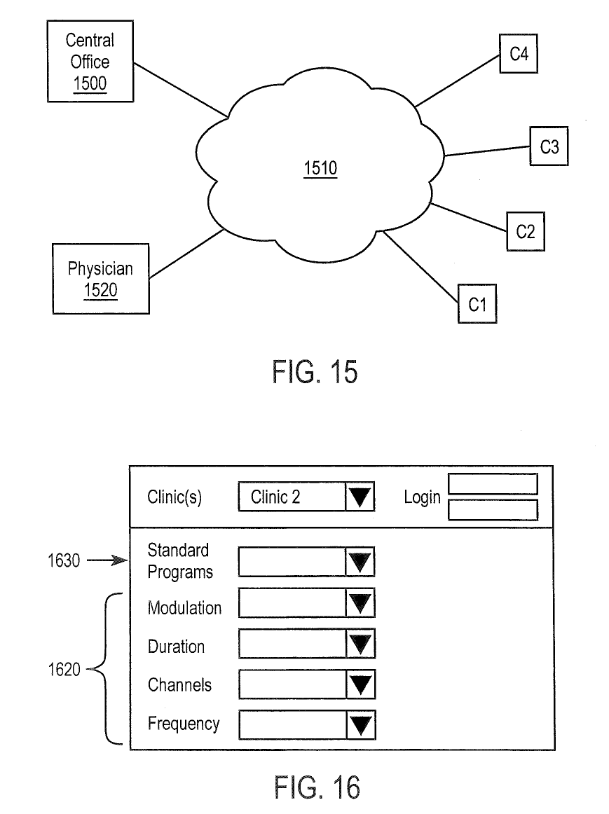

In one embodiment, as illustrated in FIG. 15, the treatment protocol is updated or revised at a central office location 1500 and then transmitted to a plurality of clinics (e.g., C1-C4) having a treatment device according to the present invention. Transmission of the treatment protocol is done, for example, via the Internet 1510 or via a wireless (e.g., cellular) network. The updated treatment protocol is then loaded into the treatment device at each corresponding clinic. In one embodiment, the treatment device includes its own network connection and automatically receives and updates the treatment protocol.

In another embodiment, a treating physician 1520 adjusts or modifies a standard treatment program according to specific needs or diagnosis of a patient. The treating physician's updated protocol is sent to the clinic, for example, via an encrypted Internet connection (e.g., PGP based encrpytion). In yet another embodiment, an example of which is illustrated in FIG. 16, a clinic includes a secure web based interface 1600 which a treating physician or technician may utilize to alter parameters of a treatment program from a remote location or in the clinic. The altered parameters may apply to a single clinic, groups of clinics, or all clinics. The altered parameters may also be specific to an individual patient, but transmitted to all clinics which the patient is authorized to attend.

The web based interface 1600 includes, for example, a physician/technician secure login, selection of a clinic 1610, frequency, modulation, channels, etc. (parameters of a treatment program) 1620, a selector for one or more standard protocols 1630, and patient identification 1640. The selections are made, for example, via pull down menus providing a range of possible entries for each selection. User defined selections may also be provided. A treatment program developed for a specific patient may also be saved as a standard treatment program for re-use with another patient requiring a similar treatment (then appearing as an optional selection in, for example, the standard programs dialog box).

Although the present invention has been described herein with reference to diabetic neuropathy induced foot pain treatments, the devices and processes described herein can be applied by the ordinarily skilled artisan to treatments for other body parts or for other ailments, particularly those related to the sympathetic nervous system.

In describing preferred embodiments of the present invention illustrated in the drawings, specific terminology is employed for the sake of clarity. However, the present invention is not intended to be limited to the specific terminology so selected, and it is to be understood that each specific element includes all technical equivalents which operate in a similar manner. For example, when describing a photon emitter, any other equivalent device, such as LEDs, lasers, light sources, radiation sources, or other devices having an equivalent function or capability, whether or not listed herein, may be substituted therewith. Furthermore, the inventors recognize that newly developed technologies not now known may also be substituted for the described parts and still not depart from the scope of the present invention. All other described items, including, but not limited to motion control devices, platforms, adjustable fixed arrays, control devices, electronics, web interface techniques, and programming, etc. should also be consider in light of any and all available equivalents.

Portions of the present invention may be conveniently implemented using a conventional general purpose or a specialized digital computer or microprocessor programmed according to the teachings of the present disclosure, as will be apparent to those skilled in the computer art.

Appropriate software coding can readily be prepared by skilled programmers based on the teachings of the present disclosure, as will be apparent to those skilled in the software art. The invention may also be implemented by the preparation of application specific integrated circuits or by interconnecting an appropriate network of conventional component circuits, as will be readily apparent to those skilled in the art based on the present disclosure.

The present invention includes a computer program product which is a storage medium (media) having instructions stored thereon/in which can be used to control, or cause, a computer to perform any of the processes of the present invention. The storage medium can include, but is not limited to, any type of disk including floppy disks, mini disks (MD's), optical discs, DVD, CD-ROMS, CDRW+/−, micro-drive, and magneto-optical disks, ROMs, RAMs, EPROMs, EEPROMs, DRAMs, VRAMs, flash memory devices (including flash cards, memory sticks), magnetic or optical cards, MEMS, nanosystems (including molecular memory ICs), RAID devices, remote data storage/archive/warehousing, or any type of media or device suitable for storing instructions and/or data.

Stored on any one of the computer readable medium (media), the present invention includes software for controlling both the hardware of the general purpose/specialized computer or microprocessor, and for enabling the computer or microprocessor to interact with a human user or other mechanism utilizing the results of the present invention. Such software may include, but is not limited to, device drivers, operating systems, and user applications. Ultimately, such computer readable media further includes software for performing the present invention, as described above.

Included in the programming (software) of the general/specialized computer or microprocessor are software modules for implementing the teachings of the present invention, including, but not limited to, control of synchronous and/or stepper motors for motion control, modulation, intensity adjustments, treatment durations, reading, storing and implementing treatment protocols and the display, storage, or communication of results according to the processes of the present invention.

The present invention may suitably comprise, consist of, or consist essentially of, any of element (the various parts or features of the invention) and their equivalents whether or not described herein. Further, the present invention illustratively disclosed herein may be practiced in the absence of any element, whether or not specifically disclosed herein. Obviously, numerous modifications and variations of the present invention are possible in light of the above teachings. It is therefore to be understood that within the scope of the appended claims, the invention may be practiced otherwise than as specifically described herein.

1.-18. (canceled)

19. A treatment method, comprising the steps of:
registering a patient;
fitting mirror image body parts of a patient into an automated photon array device configured to automatically irradiate treatment points on the patient's mirror image body parts;
treating the patient according to a treatment protocol with the automated photon array device; and
removing and debriefing the patient.

20. The treatment method according to claim 19, wherein the treatment points include treatment points on the patient's lower leg.

21. The treatment method according to claim 19, wherein the treatment points include at least three of the following treatment points:
foot dorsum, between 1st and 2nd toes, proximal to the foot's margin of web,
foot dorsum, between 2nd and third toes, proximal to the foot's margin of web,
foot dorsum between 4th and 5th toe, proximal to the foot's margin of web,
depression anterior and superior (medial side of tuberosity of calcaneum),
directly above medial malleolus tip, posterior to tibia border,
below the depression between tip of lateral malleolus and Achilles tendon,
mid-point of transverse popliteal crease, between tendons of bicepts femoris and semitendinosus, and
plantar feet.

22. The treatment method according to claim 19, wherein the automated photon array device comprises a plurality of emitter array pairs, a first emitter array of each pair set on a first treatment point, and a second emitter array of each pair set on a second treatment point, where the first treatment point and the second treatment point are mirror image points on the patient's body.

23. The treatment method according to claim 22, wherein the treatment protocol comprises a push-pull treatment program that energizes emitters in the first emitter array pair while corresponding emitters in the second emitter array pair are off and visa-versa.

24. The treatment protocol according to claim 19, wherein:
the patient's mirror image body parts comprise the patient's feet; and
the treatment protocol is configured to treat diabetic neuropathy by energizing photon arrays in the device to stimulate the patients sympathetic nervous system.

25. The treatment protocol according to claim 19, wherein at least one pair of portions of the patient's mirror image body parts are treated using a motion control device.

26.-34. (canceled)

35. A method for treating diabetic neuropathy, the method comprising:
aligning a first body part with a first photon emitter array;
aligning a second body part with a second photon emitter array, wherein the second body part is a complementary body part of the first body part;
activating the first photon emitter array; and
alternately activating the second photon emitter array according to predefined sequence, whereby only one of the first and second photon emitter arrays are activated at a particular time period; such that each photon emitter array stimulates sympathetic nerves located in body tissue next to each photon emitter array in order to treat diabetic neuropathy.

36. The method of claim 35, wherein the first and second body parts are a right foot and a left foot, respectively.

37. The method of claim 36, wherein each photon emitter array comprises four channels, and wherein for each photon emitter array:
a first channel is placed adjacent to a bottom of a respective foot;
a second channel is placed to top of the respective foot;
a third channel is placed at a rear of the respective foot; and
a fourth channel is placed behind a popliteal artery of a knee corresponding to each respective foot.

38. The method of claim 37 further comprising:
alternatively activating each of the first, second, third and fourth channels for each photon emitter array.
